(19) United States
(12) Patent Application Publication
(10) Pub. No.: US 2025/0041623 A1
(43) Pub. Date: Feb. 6, 2025

(54) THERAPEUTIC DEVICE

(71) Applicant: SHENZHEN KAIYAN MEDICAL EQUIPMENT CO., LTD, Shenzhen (CN)

(72) Inventors: ALAIN DIJKSTRA, Amstelveen (NL); CHEN SHOUFENG, Shenzhen (CN)

(73) Assignee: SHENZHEN KAIYAN MEDICAL EQUIPMENT CO., LTD, Shenzhen (CN)

(21) Appl. No.: 18/774,983

(22) Filed: Jul. 17, 2024

(30) Foreign Application Priority Data
Aug. 3, 2023 (CN) 2023220687340

(51) Int. Cl. A61N 5/06

(52) U.S. Cl. CPC A61N 5/0616; A61N 2005/0645; A61N 2005/0652; A61N 2005/0659; A61N 2005/0661; A61N 2005/0663

(57) ABSTRACT

Embodiments of the present invention provide a phototherapy patch and a method of providing phototherapy thereof. The phototherapy patch comprises a shell having a housing for a printed circuit board and a stimulation element, the shell is extended vertical upward in circular form to provide a housing for a battery. The horizontal surface of the shell has a circular opening to accommodate a power button that controls the switching of the stimulation element.

### TECHNICAL FIELD

The present invention relates generally to the field of therapeutic devices, and more specifically, the present invention relates to a portable, light-weight, hand-held therapeutic patch to provide alternate therapies for skin related diseases.

### BACKGROUND ART

In recent years, phototherapy technology has flourished in the fields of beauty and medicine. Phototherapy uses specific wavelengths of the light to act on the skin, thereby stimulating the vitality and function of cells, and improving skin problems and promoting overall health. Phototherapy beauty technology is usually performed with professional beauty equipment, and there are also some home beauty equipment or facial mask products that use red light therapy for personal care at home. However, existing light therapy products suffer from various limitations, as their structure is complex and the manufacturing cost is high, which limits the purchasing power of ordinary consumers. With the technological advancement, some technologies have begun to try to simplify the structure of light therapy devices to reduce costs, however with simplification, they suffer from performance problem, such as problems with stability and unsatisfactory phototherapy effects.

In the conventional device, the device switches the light emission of the light emitting unit by eccentric rotation. The device utilizes a rotating switch to switch off and switch-on the light emitting unit. While carrying the device, a user may accidentally touch the rotating device, resulting in a waste of power when the power supply unit is connected. The device has a light transmitting hole on the base of the phototherapy device and the sealing is not good.

Furthermore, the conventionally available phototherapy devices require a dedicated controller for regulating the functioning of LEDs, which makes the device structure complex and cumbersome.

In order to overcome the aforementioned disadvantages, the present invention provides a simple, low-cost therapeutic device with stable functions and good effects.

### OBJECTS OF THE INVENTION

Some of the objects of the invention are as follows:

An object of the present invention is to provide a therapeutic device that has a relatively very small footprint in terms of size and power consumption.

Another object of the present invention is to provide a therapeutic device that is simple, economical and has low production cost.

Another object of the present invention is to provide a therapeutic device that is convenient to hold during usage of the device.

Another object of the present invention is to provide a therapeutic device with a dedicated power button regulating the patch's ON and OFF modes, thereby eliminating the need of a controller for regulating the functioning of LEDs and making the structure simple and convenient to hold.

It is also an object of the present invention to provide a therapeutic device having a removable battery cap.

It is yet another object of the present invention to provide a therapeutic device having the light therapy lamp safeguarded by a transparent outer layer, crafted from either transparent encapsulating gel or a clear silicon shell, protecting against dust, water vapor and mechanical damage.

It is yet another object of the present invention to provide a therapeutic device with a magnetic component that enables the therapeutic device to attached on the clothes using magnets.

### SUMMARY OF THE INVENTION

According to a first aspect of the present invention, there is provided a therapeutic device. The therapeutic device comprises a shell having a housing; a printed circuit board housed in the housing, said printed circuit board has a first terminal and a second terminal on a first side; at least one stimulation element positioned on the second side of the printed circuit board; a cover layer disposed over the stimulation element; a battery; a first electrode metallic strip that connects a battery's first terminal with the first terminal on the printed circuit board; a second electrode, said second electrode is an elongated metallic strip having a push back element at a first end that connects to a battery's second terminal, and a second end of the second electrode is connected to the second terminal on the printed circuit board through a switch button; wherein the shell has an opening for housing the switch button.

In one embodiment of the invention, the at least one stimulation element is selected from a group consisting of Light Emitting Diodes (LEDs), lasers, heating elements, cooling elements, vibration elements, electrodes and combinations thereof.

In one embodiment of the invention, the stimulation element is Light Emitting Diodes (LEDs) capable of emitting electromagnetic radiation when connected to the battery. The LEDs are configured to emit electromagnetic radiation with wavelength ranges of 300 nm to 1200 nm.

In one embodiment of the invention, the shell is extended vertically to form a circular top portion.

In one embodiment of the invention, an insulation layer is provided between the battery and the shell.

In one embodiment of the invention, the cover layer is a transparent layer or a metallic layer or a ceramic layer.

In one embodiment of the invention, the therapeutic device further comprises a transparent, double-sided adhesive film to serve as an attachment means to a user's skin.

In one embodiment of the invention, the shell is fitted with a battery cover to protect the battery and the battery cover has a plurality of flanges that fits into corresponding grooves in the shell.

In one embodiment of the invention, the battery cover is circular in shape with a semi-circular projection protruding outward at one point, so as to allow a user to conveniently access the battery.

In one embodiment of the invention, the switch button connects or terminates the connection between the second electrode and the second terminal on the printed circuit board.

According to a second aspect of the present invention, there is provided a therapeutic device. The therapeutic device comprises a shell having a housing; a printed circuit board housed in the housing, said printed circuit board has a first terminal and a second terminal on a first side; at least one stimulation element positioned on the second side of the printed circuit board; a cover layer disposed over the stimulation element; a battery; a first electrode metallic strip that connects a battery's first terminal with the first terminal on the printed circuit board; a second electrode, said second electrode is an elongated metallic strip having a push back element at a first end that connects to a battery's second terminal, and a second end of the second electrode is connected to the second terminal on the printed circuit board through a switch button; wherein the shell has a circular opening for housing the switch button; wherein the shell comprises an attachment means.

In one embodiment of the invention, the stimulation element is selected from a group consisting of Light Emitting Diodes (LEDs), lasers, heating elements, cooling elements, vibration elements, electrodes and combinations thereof. The LEDs are configured to emit electromagnetic radiation with wavelength ranges of 300 nm to 1200 nm.

In one embodiment of the invention, the cover layer is a transparent layer or a metallic layer or a ceramic layer.

In one embodiment of the invention, the shell is extended vertically to form a circular top portion.

In one embodiment of the invention, the attachment means is a magnetic component that binds to a magnetic or metallic components on a user's cloth.

In one embodiment of the invention, the attachment means is a magnetic component that binds to a magnetic or a metallic component on a user's cloth.

In one embodiment of the present invention, the attachment means is a clip, a pin or a binder.

In one embodiment of the invention, the shell is fitted with a battery cover to protect the battery, and the battery cover has a plurality of flanges that fits into corresponding grooves in the shell.

In one embodiment of the invention, the battery cover is circular in shape with a semi-circular projection protruding outward at one point, so as to allow a user to conveniently access the battery.

In one embodiment of the invention, the switch button connects or terminates the connection between the second electrode and the second terminal on the printed circuit board.

According to a third aspect of the present invention, there is provided a method for providing a therapy to a user. The method comprises: providing a therapeutic device comprising a shell having a housing for a printed circuit board, at least one stimulation element and a battery, said shell is extended vertically in circular shape to provide a housing for a battery, said shell has a opening on the horizontal surface to accommodate a power button that controls the switching of the at least one stimulation element; putting the battery inside the therapeutic device; applying a transparent adhesive tape on the therapeutic device; affixing the therapeutic device on a user's body; switching ON the power button for providing treatment to the user.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of the specification, the phrase “communication network” refers to a group of several connected devices including computing devices (such as desktops, mobile handheld devices, tablet PCs, notebooks, etc.), local and remotely located servers (such as web servers, application servers, database servers, Application Program Interface (API) servers, load balancers, compute nodes, and the like), routers, antennas, modems, multiplexers, demultiplexers, and the like. In that regard, the aforementioned connected devices may be able to exchange data signals through wired and/or wireless means as per several combinations of several different communication protocols such as 802.11 (Wi-Fi), 802.3 (Ethernet), Bluetooth, NFC, ZigBee and 3GPP protocols such as HSPA, HSDPA, LTE, GSM, CDMA, WLL and the like.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, a “diaphanous material” is a material that allows at least a portion of one or more forms (such as Infrared, Ultraviolet, X-Rays, Visible Light, Microwaves, Radio Waves, etc.) of electromagnetic radiation to pass through them. The diaphanous materials can be transparent (allowing the one or more forms of the electromagnetic radiation to pass through with minimal scattering) or translucent (allowing the one or more forms of the electromagnetic radiation to pass through with appreciable diffusion or scattering). Diaphanous materials can be dense, like glass, or have an open structure, like wire mesh or a woven fabric.

In the context of the specification, the term “historical” in execution of a command refers to anything pertaining to a time instant(s) that is earlier than a time instant of an initiation of the command.

In the context of the specification, the term, “real-time”, refers to without intentional delay, given the processing limitations of hardware/software/firmware involved and the time required to accurately measure/receive/process/transmit data as practically possible.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a top view of a therapeutic device, in accordance with an embodiment of the present invention;

FIG. 1B illustrates a top perspective view of a therapeutic device having a heart shaped shell in accordance with an embodiment of the present invention;

FIG. 1C illustrates a bottom perspective view of the therapeutic device of FIG. 1A;

FIG. 2 illustrates an exploded view of the therapeutic device, in accordance with an embodiment of the present invention;

FIG. 3A illustrates a therapeutic device with a magnetic component acting as a fastener, in accordance with an embodiment of the present invention;

FIG. 3B illustrates the therapeutic device with the magnetic component worn by a user, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is, therefore, not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a therapeutic device. The therapeutic device is a phototherapy device in form of a patch, a type of sticker employing a stimulation element, such as Light Emitting Diodes (LEDs), lasers, heating elements, cooling elements, vibratory elements, electrodes etc. The device comprises a metallic or non-metallic shell having a circuit board and a stimulation element installed on the circuit board. The stimulation element is connected to a power source. The device has a dedicated power button that regulates the device's ON and OFF modes. The attachment of the therapeutic device to the user's skin is facilitated by an adhesive film. The therapeutic device further comprises a magnetic component that is used to attach the device to the user's clothes using a magnet.

Several additional functionalities may also be added to the therapeutic device. For example, an opening is provided on the back side of the shell and the power button is detachably installed on the circuit board through the opening. The therapeutic device has a detachable battery cover that is used to fit over the opening. The therapeutic device has a transparent layer arranged inside the outer film of the stimulating agent. The transparent layer is crafted from either transparent encapsulating gel or a clear silicon shell, thereby protecting against dust, water vapor and mechanical damage. The stimulating agent is equipped with a transparent layer outside, which can prevent the light of the LEDs being blocked. The transparent layer is provided with a film which is directly attached to the user's skin. Moreover, activation and control of specific stimulation element may be based upon feedback received from several sensors such as pressure sensors (for ascertaining contact with a body portion of the user) and temperature sensors (for maintaining the temperature of heating and/or cooling elements within permissible limits). Electrical power to operate the therapeutic device may be provided through an onboard rechargeable battery that may be charged using a power cable.

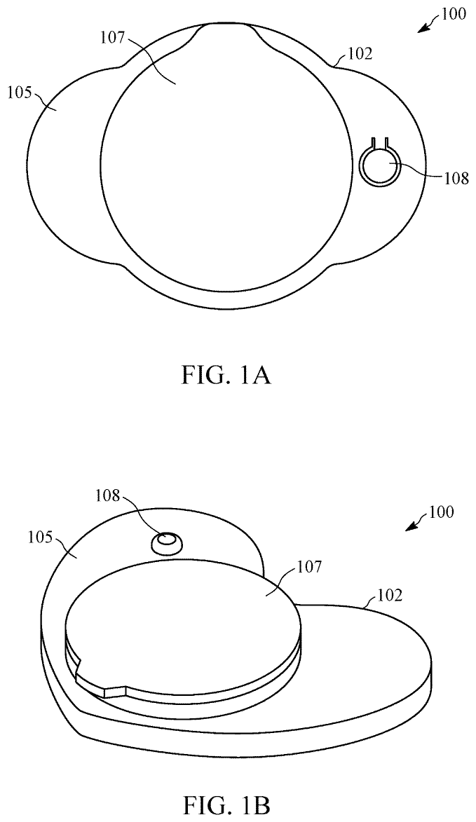

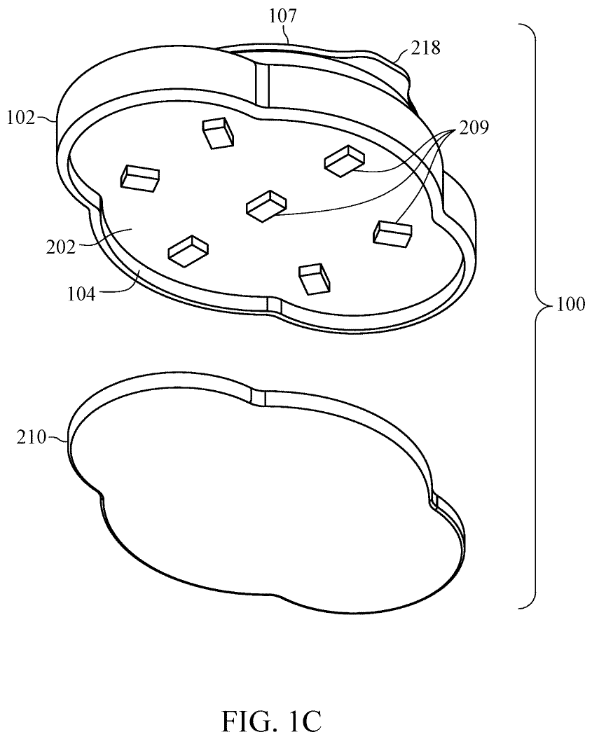

Several embodiments of the present invention will now be described in detail with references to FIGS. 1A-3B.

FIG. 1A illustrates a top view of a therapeutic device or a therapeutic patch 100 (hereinafter also referred to as “the patch 100”), in accordance with an embodiment of the present invention. FIG. 1B illustrates a top view of a therapeutic device 100 having a heart shaped shell in accordance with an embodiment of the present invention. FIG. 1C illustrates a bottom perspective view of the phototherapy device 100 of FIG. 1A. The patch 100 comprises a shell 102 having a housing 104 that encapsulates several different elements of the patch 100. Different components housed by the housing 104 comprises a circuit board, a stimulation element, a pair of electrodes, a switch button and a power supply. The shell 102 is made of metallic or non-metallic component and may be in any shape. The shell 102 is hollow inside to provide the housing 104 to accommodate different components. The top side of the shell 102 is extended vertically in cylindrical form to form a top portion 106 to accommodate a button cell or a rechargeable battery. A battery cover 107 is used to cover the rechargeable battery. The battery cover 107 has flanges at the bottom that snuggly fits into corresponding grooves in the top portion 106 of the shell 102. The shell 102 has a circular opening at horizontal surface 105 to accommodate a power button 108 that regulates the switch ON and OFF the power supply from the rechargeable battery to the stimulation element.

The patch 100 has an attachment means that is used to fix the patch 100 on to a user's skin or a specific part of human body to provide therapy to the corresponding part. The attachment means may be in form of an adhesive tapes that sticks to the user's skin or a magnet in the patch 100 that attaches on the corresponding attachment means on the user's clothes. The patch has a dedicated power button 108 that controls the switch modes of the patch 100 thereby eliminating the complex controller structure. When the patch 100 is applied on the user's body, the light therapy effect is achieved by specific light emitted by the stimulation element, such as LEDs.

In several embodiments of the invention, the stimulation elements may be selected from a group consisting of Light Emitting Diodes (LEDs), lasers, heating elements, cooling elements, vibration elements, electrodes etc.

In the case of the stimulation element being the LED or the laser, the stimulation element may be emitting electromagnetic radiation. In the case of the stimulation element being the vibration element, the stimulation element may be a vibrating head connected to an eccentric mass rotating motor or a linear resonant motor. In the case of the stimulation element being the heating element, the stimulation element may be selected from a group consisting of metal heating elements, ceramic heating elements, semiconductor heating elements, thick film heating elements, polymer-based heating elements, composite heating elements, and combination heating elements. In the case of the stimulation element being the cooling element, the stimulation element may be a thermoelectric cooler, also known as a Peltier heat pump. In the case of the stimulation element being an electrode, the stimulation element may be embodied as an open-ended conductor. The stimulation element may then be able to provide Transcutaneous Electrical Nerve Stimulation (TENS), Electronic Muscle Stimulation (EMS), and Microcurrent Electrical Therapy (MET) to the body of a user.

TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

In an embodiment of the present invention, the width of the therapeutic device is in range of 5 mm to 20 mm.

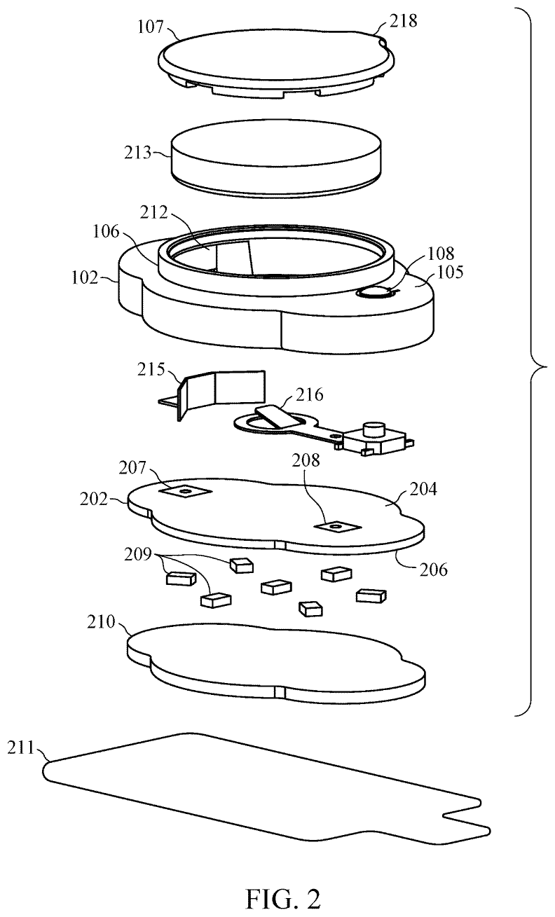

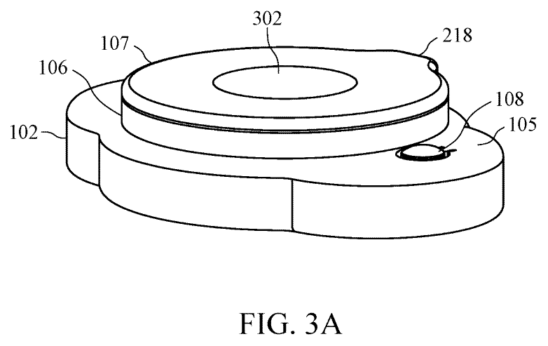

FIG. 2 illustrates an exploded view of the therapeutic device or therapeutic patch, in accordance with an embodiment of the present invention. The patch 100 comprises a shell 102 having a housing 104 that encapsulates several different elements of the patch 100. Different components housed by the housing 104 comprises a circuit board, a stimulation element, a pair of electrodes, a switch button and a power supply. The shell 102 is made of metallic or non-metallic component and may be in any shape. The shell 102 is hollow inside to provide the housing 104 to accommodate different components. The top side of the shell 102 is extended vertically in cylindrical form to form a top portion 106, the top portion 106 is circular in shape.

A printed circuit board 202 with complementary shape as that of the shell 102 is placed inside the housing 104 of the shell 102. The printed circuit board 202 has a first side 204 and a second side 206, the printed circuit board 202 when inserted inside the housing 104 of the shell 102 has the first side 204 facing inward and the second side 206 facing outward. The first side 204 has a positive terminal 208 (a second terminal) and a negative terminal 207 (a first terminal) to provide connection with a power source. The second side 206 of the printed circuit board 202 has a stimulation element 209 fixed on the printed circuit board 202.

The stimulation element 209 is in electrical connection with the positive terminal 208 and negative terminal 207 on the first side 204 of the printed circuit board 202. A diaphanous cover layer 210 is disposed over the stimulation element 209. The diaphanous cover layer 210 fixes the printed circuit board 202 for preventing any lateral movement. The diaphanous cover layer 210 protects the printed circuit board 202 from dust, water vapor and mechanical damage. The diaphanous cover layer 210 being transparent does not have effect on transmission of radiation from the stimulation element 209. The diaphanous cover layer 210 is a transparent encapsulating glue for encapsulating the printed circuit board 202 and the attached devices. The diaphanous cover layer 210 may be a transparent silicone cover.

The diaphanous cover layer 210 is covered with a transparent film 211. The transparent film 211 is a skin-friendly, transparent, double-sided adhesive tape. The transparent film 211 being adhesive serves as an attachment means to a user's skin and can directly stick the patch 100 on the skin for use.

The top portion 106 of the shell 102 has a circular opening 212 to provide space for accommodating a rechargeable battery 213 that act as a power source to provide current to the stimulation element 209. The battery 213 is covered with a detachable battery cover 107. The battery cover 107 comprises a plurality of flanges extended downward from the battery cover such that the battery cover when placed over the top portion 106 of the shell 102, the flanges get snuggly fitted into the corresponding grooves in the top portion 106 of the shell 102. The shape of the battery cover 107 is circular with a semi-circular projection 218 protruding outward at one point of the battery cover 107. The semi-circular projection 218 allows a user to conveniently open the battery cover 107 in order to access the battery 213.

To provide electric connection between the battery 213 and the stimulation element 209, a pair of electrodes are provided: a positive electrode 216 (a second electrode) that connects the positive terminal (the second terminal) of the battery 213 with the positive terminal 208 (the second terminal) on the first side 204 of the printed circuit board 202; and a negative electrode 215 (a first electrode) that connects the negative terminal (first terminal) of the battery 213 with the negative terminal 207 (the first terminal) on the first side 204 of the printed circuit board 202.

The negative electrode (first electrode) 215 is a metallic strip connected to the side of the top portion 106 of the shell 102, such that when battery 213 is inserted in the top potion 106 of the shell 102, the metallic strip is in connection with the negative terminal of the battery 213. The second end of the metallic strip or negative electrode 215 is connected to the negative terminal 207 on the first side of the printed circuit board.

The positive electrode (second electrode) 216 is a metallic strip placed over the first side 204 of the printed circuit board 202. The positive electrode 216 is a push back strip which when the battery 213 is inserted comes in contact with the positive terminal of the battery 213. The positive electrode 216 is connected to the positive terminal 208 on the first side 204 of the printed circuit board 202 through a switch or a power button 108. The power button 108, when pressed, establishes the continuous connection between the positive electrode 216 and the positive terminal 208 on the first side 204 of the printed circuit board 202. The power button 108 is accessible to the user for operating through a circular opening at horizontal surface 105.

In an embodiment, the dedicated power button 108 eliminates the need of controller in the device for regulating the functioning of the plurality of phototherapy lamp beads.

In an embodiment of the present invention, an insulation layer is provided between the electrical components of the therapeutic device and the shell.

In an embodiment of the present invention, the width of the therapeutic device is in range of 5 mm to 20 mm.

In several embodiments of the invention, the stimulation element 209 may be selected from a group consisting of Light Emitting Diodes (LEDs), lasers, heating elements, cooling elements, vibration elements, electrodes etc.

In the case of the stimulation clement 209 being the LED or the laser, the stimulation clement 209 may be emitting electromagnetic radiation. In the case of the stimulation element 209 being the vibration element, the stimulation element 209 may be a vibrating head connected to an eccentric mass rotating motor or a linear resonant motor. In the case of the stimulation element 209 being the heating element, the stimulation element 209 may be selected from a group consisting of metal heating elements, ceramic heating elements, semiconductor heating elements, thick film heating elements, polymer-based heating elements, composite heating elements, and combination heating elements. In the case of the stimulation element being the cooling element, the stimulation element may be a thermoelectric cooler, also known as a Peltier heat pump. In the case of the stimulation element 209 being an electrode, the stimulation element may be embodied as an open-ended conductor. The stimulation clement 209 may then be able to provide Transcutaneous Electrical Nerve Stimulation (TENS), Electronic Muscle Stimulation (EMS), and Microcurrent Electrical Therapy (MET) to the body of a user.

TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

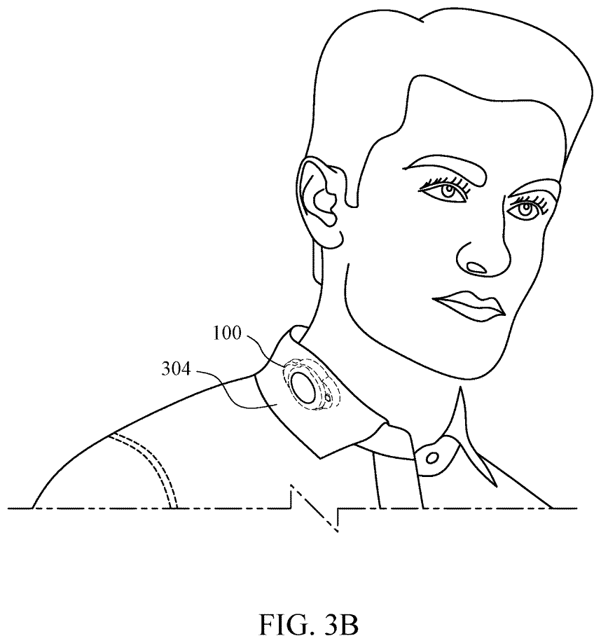

FIG. 3A illustrates a therapeutic device or therapeutic patch with a magnetic component that acts as an attachment means, in accordance with an embodiment of the present invention. The patch 100 has a magnetic component 302 embedded in it or attached to the battery cover 107. The magnetic component 302 serves as an attachment means to provide coupling of device on to user's clothes. The magnetic component 302 is either present at the top of the patch 100, in the battery cover 107 or present in the side body of the patch 100. The magnet binds with the corresponding magnet/metallic component present on the user's clothes and the device is fixed on the user's clothes.

FIG. 3B illustrates the phototherapy patch 100 with the magnet worn by a user, in accordance with an embodiment of the present invention. Referring to FIG. 3B, the phototherapy patch 100 is attached to the user's collar 304 through a magnetic attachment means. A magnet or a metallic component is fixed on the user's collar 304. The magnetic component 302 in the phototherapy patch binds to the magnet or metallic component on the user's collar, thereby fixing the phototherapy patch on the user's neck.

The embodiments of the present invention as presented above offer several advantages. For instance, the therapeutic device 100 is compact in construction and can be further collapsed for convenient storage and handling. The design is simple in construction and does not require manufacturing of complex geometries. The use of dedicated power button 108 in the therapeutic device 100 eliminates the need of a separate controller for regulating the functioning of the stimulation element 209. The power button 108 is easily accessible by the user, therefore eliminates the accidental switching on the therapeutic device 100 in order to avoid wastage of power. The power button 108 is accessible through a circular opening on the horizontal surface 105 of the shell 102, therefore eliminating the need of separate switch and making the structure simple. Moreover, the shape of the therapeutic device 100 is designed with a projection 218 on circular battery cover 107 so that a user can easily wrap around his fingers on the therapeutic device 100 and handle the therapeutic device 100 conveniently. Moreover, the therapeutic device 100 is provided with a magnetic component 302 as an attachment means that helps in attaching the therapeutic device 100 on a user's clothes through corresponding attachment means.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapeutic device, comprising:
a shell having a housing;
a printed circuit board housed in the housing, the printed circuit board has a first terminal and a second terminal on a first side;
at least one stimulation element positioned on a second side of the printed circuit board;
a cover layer disposed over the at least one stimulation element;
a battery;
a first electrode metallic strip that connects a battery's first terminal with the first terminal on the printed circuit board;
a second electrode, the second electrode is an elongated metallic strip having a push back element at a first end that connects to a battery's second terminal, and a second end of the second electrode is connected to the second terminal on the printed circuit board through a switch button;
wherein the shell has an opening for housing the switch button.

2. The therapeutic device of claim 1, wherein the at least one stimulation element is selected from a group consisting of Light Emitting Diodes (LEDs), lasers, heating elements, cooling elements, vibration elements, electrodes and combinations thereof.

3. The therapeutic device of claim 2, wherein the Light Emitting Diodes (LEDs) emit electromagnetic radiation when connected to the battery.

4. The therapeutic device of claim 1, wherein the shell is extended vertically to form a circular top portion.

5. The therapeutic device of claim 1, wherein the shell is made of a metallic component or a non-metallic component.

6. The therapeutic device of claim 1, wherein an insulation layer is provided between shell, the printed circuit board, the first electrode and the second electrode.

7. The therapeutic device of claim 1, wherein the cover layer is a transparent layer, a metallic layer or a ceramic layer.

8. The therapeutic device of claim 1, wherein the device further comprises a transparent, double-sided adhesive film to serve as an attachment to a user's skin.

9. The therapeutic device of claim 1, wherein the shell is fitted with a battery cover to protect the battery, the battery cover has a plurality of flanges that fit into corresponding grooves in the shell.

10. The therapeutic device of claim 9, wherein the battery cover is circular in shape with a semi-circular projection protruding outward at one point, so as to allow a user to access the battery.

11. The therapeutic device of claim 1, wherein the switch button connects or terminates the connection between the second electrode and the second terminal on the printed circuit board.

12. A phototherapy patch, comprising:
a shell having a housing;
a printed circuit board housed in the housing, the printed circuit board has a first terminal and a second terminal on a first side;
at least one stimulation element positioned on a second side of the printed circuit board;
a cover layer disposed over the at least one stimulation element;
a battery;
a first electrode metallic strip that connects a battery's first terminal with the first terminal on the printed circuit board;
a second electrode, the second electrode is an elongated metallic strip having a push back element at a first end that connects to a battery's second terminal, and a second end of the second electrode is connected to the second terminal on the printed circuit board through a switch button;
wherein the shell has an opening for housing the switch button;
wherein the shell comprises an attachment component.

13. The therapeutic device of claim 12, wherein the at least one stimulation element is selected from a group consisting of Light Emitting Diodes (LEDs), lasers, heating elements, cooling elements, vibration elements, electrodes, and combinations thereof.

14. The therapeutic device of claim 12, wherein the cover layer is a transparent layer, a metallic layer or a ceramic layer.

15. The therapeutic device of claim 12, wherein the shell is extended vertically to form a circular top portion.

16. The therapeutic device of claim 12, wherein the attachment component is a magnetic component that binds to a magnetic or a metallic components on a user's cloth.

17. The therapeutic device of claim 12, wherein the attachment component is a clip, a pin or a binder.

18. The therapeutic device of claim 12, wherein the shell is fitted with a battery cover to protect the battery, the battery cover has a plurality of flanges that fit into corresponding grooves in the shell.

19. The therapeutic device of claim 18, wherein the battery cover is circular in shape with a semi-circular projection protruding outward at one point, so as to allow a user to access the battery.

20. The therapeutic device of claim 12, wherein the switch button connects or terminates the connection between the second electrode and the second terminal on the printed circuit board.

21. A method for providing a therapy to a user, the method comprising:
providing a therapeutic device comprising a shell having a housing for a printed circuit board, at least one stimulation element and a battery, the shell is extended vertically in circular shape to provide a housing for a battery, the shell has an opening on the horizontal surface to accommodate a power button that controls the switching of the at least one stimulation element;
putting the battery inside the therapeutic device;
applying a transparent adhesive tape on the therapeutic device;
affixing the therapeutic device on a user's body;
switching ON the power button for providing treatment to the user.
